(19) United States
(12) Patent Application Publication
(10) Pub. No.: US 2006/0002885 A1
(43) Pub. Date: Jan. 5, 2006

(54) Use of combinations of active ingredients of one or more bioquinones and one or more isoflavones for improving the contours of the skin

(75) Inventors: Heiko Mielke, Neu Wulmstorf (DE); Soren Jaspers, Schenefeld (DE); Gunhild Hamer, Hamburg (DE); Christopher Mummert, Bienenbuttel (DE); Jens Schulz, Schenefeld (DE); Stefan Gallinat, Wedel (DE); Kirsten Venzke, Emden (DE); Frank Schwanke, Hamburg (DE)

Correspondence Address: GREENBLUM & BERNSTEIN, P.L.C., 1950 ROLAND CLARKE PLACE, RESTON, VA, 20191, US

(73) Assignee: BEIERSDORF AG, Hamburg (DE)

(21) Appl. No.: 10/935,246

(22) Filed: Sep. 8, 2004

(30) Foreign Application Priority Data
Jul. 2, 2004 (DE) 10 2004 032 837.4

(51) Int. Cl. A61K 8/97; A61K 36/48

(52) U.S. Cl. 424/74; 424/757

(57) ABSTRACT

A method of tightening and/or firming the skin and a method for the treatment and/or prophylaxis of cellulite, wherein the methods comprise applying to at least a part of the skin a cosmetic or dermatological O/W emulsion which comprises one or more bioquinones and one or more isoflavonoids.

The present application claims priority under 35 U.S.C. § 119 of German Patent Application No. 10 2004 032837.4, filed Jul. 2, 2004, the entire disclosure of which is incorporated by reference herein.

The present invention relates to cosmetic or dermatological preparations containing active substances for the care and protection of the skin, in particular sensitive skin and very particularly skin aged or aging by intrinsic and/or extrinsic factors and the use of such active substances and combinations of such active substances in the field of cosmetic and dermatological skin care.

Cosmetic skin care means primarily strengthening or rebuilding the natural function of the skin as a barrier against environmental influences (e.g., dirt, chemicals, microorganisms) and against the loss of endogenous substances (e.g., water, natural fats, electrolytes).

If this function is impaired, an intensified absorption of toxic or allergenic substances or an attack by microorganisms leading to toxic or allergic skin reactions can occur.

The aim of skin care is furthermore to compensate for the loss by the skin of lipids and water caused by daily washing. This is particularly important if the natural regenerative ability is insufficient. Moreover, skin care products should protect against environmental influences, in particular sun and wind, and delay skin aging.

Chronological skin aging is caused, e.g., by endogenous, genetically determined factors. The following structural damage and functional disorders, which can also come under the term “senile xerosis”, can occur, e.g., in the epidermis and the dermis due to aging:
(a) Dryness, roughness and formation of small lines due to dryness,
(b) Itching and
(c) Reduced regreasing by sebaceous glands (e.g., after washing).

Exogenous factors, such as UV light and chemical noxae, can have a cumulative effect and, e.g., accelerate or supplement the endogenous aging processes. In the epidermis and dermis, in particular due to exogenic factors, e.g., the following structural damage and functional disorders occur in the skin that go beyond the extent and quality of the damage with chronological aging:
(d) Visible vascular dilation (telangiectases, couperosis);
(e) Flabbiness and formation of wrinkles;
(f) Local hyperpigmentation, hypopigmentation and abnormal pigmentation (e.g., senile keratoses) and
(g) Increased susceptibility to mechanical stress (e.g., cracking).

The present invention relates in particular to products for the care of skin that has aged naturally, and to the phenomena listed under a), e) and g).

Products for the care of flaccid, in particular aged skin are known per se. They contain, e.g., retinoids (vitamin A acid and/or derivatives thereof or vitamin A and/or derivatives thereof. However, the degree of their effect on structural damage is limited. Moreover, in product development there are considerable difficulties in stabilizing the active substances sufficiently against oxidative decay. Moreover, the use of products containing vitamin A acid often causes severe erythematous skin irritations. Retinoids can therefore be used only in low concentrations.

Flaccid skin is often also associated with an accompanying phenomenon of excess weight and/or so-called cellulite frequently associated therewith.

The body-consciousness of consumers has increased significantly in recent years. In this connection, in addition to cleansing and care applications, measures are also increasingly being taken to improve the silhouette of the body. Cellulite—a widespread phenomenon—is thereby of central importance. The visible appearance of cellulite is based on an increase of fatty bodies in the subcutis (subcutaneous fatty tissue), a weakness of the connective tissue and a reduction of the perfusion ratios in the blood stream and lymphatic tract. The cause is thus a partially constitutional weakening of the connective tissue with the simultaneous occurrence of enlarged fat cell chambers as a result of excess weight, an unbalanced diet, lack of exercise. The formation of cellulite can furthermore be attributed to increased permeability of the capillary walls which permits the penetration of water into the connective tissue.

In addition, there can be a local testosterone deficiency at the affected areas of the skin. In any case, cellulite is a phenomenon that is virtually never observed in men.

The object of the present invention was therefore to find ways to avoid the disadvantages of the prior art. In particular it was an object of the present invention to make available preparations that can effect an advantageous tightening of flaccid skin.

Surprisingly, it has been found that the use of cosmetic or dermatological preparations in the form of O/W emulsions with a content of
(a) one or more bioquinones and
(a) one or more isoflavonoids for tightening and/or strengthening the skin overcomes the disadvantages of the prior art.

According to the invention, it is also advantageous to use cosmetic or dermatological preparations in the form of O/V emulsions with a content of
(a) one or more bioquinones and
(a) one or more isoflavonoids for the cosmetic and/or dermatological treatment and/or prophylaxis of cellulite.

Ubiquinones are characterized by the structural formula 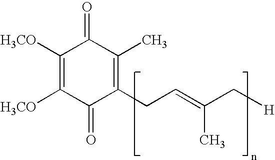
and represent the most widespread and thus most investigated bioquinones. Ubiquinones are referred to as Q-1, Q-2, Q-3, etc., depending on the number of isoprene units linked in the side-chain, or as U-5, U-10, U-15, etc., depending on the number of carbon atoms. They preferentially occur with certain chain lengths, e.g., in some microorganisms and yeasts where n=6. In the case of most mammals, including humans, Q10 predominates.

Coenzyme Q10, for example, is characterized by the following structural formula:

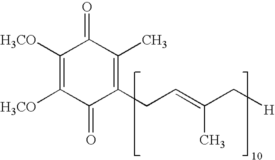

Ubiquinones serve organisms as electron transfer agents in the respiratory chain. They are located in the mitochondria where they render possible the cyclic oxidation and reduction of the substrates of the citric acid cycle.

Plastoquinones have the general structural formula

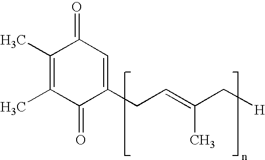

They can be isolated from chloroplasts and they play a role as redox substrates in photosynthesis in the cyclic and non-cyclic electron transfer, being reversibly converted to the corresponding hydroquinones (plastoquinol). Plastoquinones differ in the number n of isoprene radicals and are named accordingly, e.g., PQ-9 (n=9). Furthermore, other plastoquinones with varying substituents on the quinone ring exist.

Japanese published patent application 58,180,410 describes the suitability of coenzyme Q10 for cosmetics. It is said to activate skin cell metabolism and to suppress oxidation. As a result, coenzyme Q10 has an important function in the prevention of skin damage by UV rays and in the prevention of skin aging.

International patent application documents WO95/26180, WO95/26181 and WO95/26182 relate to cosmetic and dermatological preparations with a content of ubiquinones or active substance combinations with ubiquinones, where these preparations can also be used against senile xerosis and skin aging. Although these preparations are occasionally present as O/W emulsions as well, they have a much lower glycerin content than is advantageous within the scope of the technical teaching submitted here.

Also known, from DE-A-33 09 850, are cosmetic preparations with coenzyme Q10 which are suitable for the treatment of skin diseases, for the prophylaxis of dystrophic and dysmetabolic conditions of the skin and for use in cases of chemical and physical respiratory damage or in cases of delayed respiration associated with age and wear.

However, none of these documents could prepare the way for the present invention.

The bioquinone preferred according to the invention is the coenzyme Q10. It is advantageous in the finished preparations to select concentrations of 0.000,001-5% by weight of one or more bioquinones, preferably coenzyme Q10, based on the total weight of the preparations.

Isoflavones are the group belonging to the flavonoids, sometimes also referred to as isoflavonoids, of plant dyes usually yellowish in color, which are derived from isoflavone. The unsubstituted basic skeleton, the actual isoflavone (3-phenyl chromone, 3-phenyl-4H-1-benzopyran-4-one) occurs in types of clover.

Some of the better known isoflavones are daidzeïn (4′,7-dihydroxyisoflavone), as 7-O-glucoside daidzin in soybean flour; genistein (4′,5,7-trihydroxyisoflavone) from soybeans and red clover; prunetin (4′,5-dihydroxy-7-methoxyisoflavone) from the bark of plum trees; biochanin A (5,7-dihydroxy-4′-methoxyisoflavone) from chickpeas, red clover and other types of clover; orobol (3′,4′,5,7-tetrahydroxyisoflavone); santal (3′,4′,5-trihydroxy-7-methoxyisoflavone) from sandalwood, redwood and other woods; pratenseïn (3′,5,7-trihydroxy-4′-methoxyisoflavone) from fresh red clover or meadow clover. Some of these isoflavones occurring in types of clover and legumes such as lucerne show an estrogen effect on grazing animals and can possibly lead to reproductive disorders among them.

Substitution schemes of a number of naturally occurring isoflavones are given below:

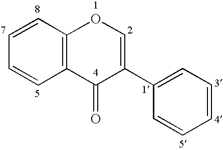

| | 5 | 7 | 3′ | 4′ | CAS-No. |
|---|---|---|---|---|---|
| Isoflavone | H | H | H | H | 574-12-9 |
| Daidzeïn | H | OH | H | OH | 486-68-8 |
| Genistein | OH | OH | H | OH | 446-72-0 |
| Genistin | OH | OH | H | OH | 446-72-0 |
| Prunetin | OH | OCH₃ | H | OH | 552-59-0 |
| Biochanin A | OH | OH | H | OCH₃ | 491-80-5 |
| Orobol | OH | OH | OH | OH | 480-23-9 |
| Santal | OH | OCH₃ | OH | OH | 529-60-2 |
| Pratenseïn | OH | OH | OH | OCH₃ | 2284-31-3 |

Suitable isoflavones to be used according to the invention are preferably those listed in the above table.

Of these in turn preference is given to genistein.

Genistein, 5,7-dihydroxy-3-(4-hydroxyphenyl)-4H-1-benzopyran-4-one, 4′,5,7-trihydroxyisoflavone, also called differenol A, prunetol and sophoricol, has the following structure:

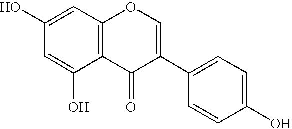

Genistein is a secondary metabolite of plants (legumes, papilionoides, rosaceous plants), but was also found in cultures of microorganisms (actinomycetes, aspergillus, mycobacteria). Weak estrogen and antibacterial effects were described.

Cosmetic or dermatological preparations according to the invention preferably contain 0.001-10% by weight, particularly preferably 0.01-1% by weight, of one or more isoflavonoids based on the total composition of the preparations.

Preferably the weight ratios of one or more isoflavonoids to one or more bioquinones in the active substance combinations and preparations containing such active substance preparations, are selected from the range of 50:1 to 1:50, advantageously from the range of 10:1 to 1:50, particularly advantageously 2:1 to 1:2.

In particular it is thereby advantageous if these extracts are additionally characterized by a content of saponins, since the effects to be achieved in this case are particularly marked. The content of saponins should thereby lie in a concentration range of 0.001-2% by weight, preferably 0.02-0.04% by weight, based on the total composition of the cosmetic preparation.

An advantageous embodiment of the present invention can thereby be that the isoflavonoid(s) is/are used as an isoflavonoid-containing extract in the form of a soy extract that, in addition to isoflavonoids, preferably contains genistein, 5-20% by weight of saponins, particularly advantageously 10-18% by weight of saponins, in each case based on the total weight of the extract.

The use of these preparations leads to an improvement of the skin contours and to an increased elasticity, in particular in the areas of the body with weak connective tissue, such as, e.g., on the legs, on the stomach and on the buttocks. These skin-tightening and skin-firming effects are associated through an improvement of the cell metabolism with a reduction of orange-peel skin, thus, e.g., with the reduction of pitting of the skin on the upper thigh area (“saddlebags”).

According to the invention it is in particular extremely advantageous to use the active substance used according to the invention or cosmetic or topical dermatological preparations with an effective content of an active substance used according to the invention for cosmetic or dermatological treatment or prophylaxis of undesirable skin conditions.

According to the invention customary antioxidants can be used in preparations that contain the active substance combinations according to the invention.

The antioxidants are advantageously chosen from the group consisting of amino acids (e.g., glycine, histidine, tyrosine, tryptophan) and derivatives thereof, imidazoles (e.g., urocanic acid) and derivatives thereof, peptides, such as D,L-carnosine, D-carnosine, L-carnosine and derivatives thereof (e.g., anserine), carotenoids, carotenes (e.g., α-carotene, β-carotene, lycopene) and derivatives thereof, lipoic acid and derivatives thereof (e.g., dihydrolipoic acid), aurothioglucose, propylthiouracil and other thiols (e.g., thioredoxin, glutathione, cysteine, cystine, cystamine and the glycosyl, N-acetyl, methyl, ethyl, propyl, amyl, butyl and lauryl, palmitoyl, oleyl, γ-linoleyl, cholesteryl and glyceryl esters thereof) and salts thereof, dilauryl thiodipropionate, distearyl thiodipropionate, thiodipropionic acid and derivatives thereof (esters, ethers, peptides, lipids, nucleotides, nucleosides and salts) and sulfoximine compounds (e.g., buthionine sulfoximines, homocysteine sulfoximine, buthionine sulfones, penta-, hexa-, heptathionine sulfoximine) in very low tolerated doses (e.g., pmol to pmol/kg), and (metal) chelating agents (e.g., α-hydroxy fatty acids, palmitic acid, phytic acid, lactoferrin), α-hydroxy acids (e.g., citric acid, lactic acid, malic acid), humic acid, bile acid, bile extracts, bilirubin, biliverdin, EDTA, EGTA and derivatives thereof, unsaturated fatty acids and derivatives thereof (e.g., γ-linolenic acid, linoleic acid, oleic acid), folic acid and derivatives thereof, alaninediacetic acid, flavonoids, polyphenols, catechols, vitamin C and derivatives (e.g., ascorbyl palmitate, Mg ascorbyl phosphate, ascorbyl acetate) tocopherols and derivatives (e.g., vitamin E acetate), and coniferyl benzoate of benzoin resin, rutinic acid and derivatives thereof, ferulic acid and derivatives thereof, butylated hydroxytoluene, butylated hydroxyanisole, nordihydroguaiacic acid, nordihydroguaiaretic acid, trihydroxybutyrophenone, uric acid and derivatives thereof, mannose and derivatives thereof, zinc and derivatives thereof (e.g., ZnO, ZnSO₄), selenium and derivatives thereof (e.g., selenomethionine), stilbenes and derivatives thereof (e.g., stilbene oxide, trans-stilbene oxide) and the derivatives (salts, esters, ethers, sugars, nucleotides, nucleosides, peptides and lipids) of these referenced active substances that are suitable according to the invention.

The amount of antioxidants (one or more compounds) in the preparations is preferably 0.001 to 30% by weight, particularly preferably 0.05-20% by weight, in particular 1-10% by weight, based on the total weight of the preparation.

The prophylaxis or the cosmetic or dermatological treatment with the active substance used according to the invention or with the cosmetic or topical dermatological preparations with an effective content of active substance used according to the invention is carried out in the usual manner by applying the active substance used according to the invention or the cosmetic or topical dermatological preparations with an effective content of active substance used according to the invention to the affected areas of the skin.

Emulsions according to the invention for the purposes of the present invention, e.g., in the form of a cream, a lotion or a cosmetic milk, are advantageous and contain, e.g., fats, oils, waxes and/or other fatty substances, and water and one or more emulsifiers as are customarily used for this type of formulation.

It is also possible and advantageous for the purposes of the present invention to incorporate the active substance used according to the invention in aqueous systems or surfactant preparations for cleansing the skin and the hair.

One of skill in the art is of course aware that demanding cosmetic compositions are mostly inconceivable without the customary auxiliaries and additives. Examples thereof include builders, fillers, perfume, dyes, emulsifiers, additional active substances, such as vitamins or proteins, light-protection agents, stabilizers, insect repellents, alcohol, water, salts and antimicrobially, proteolytically or keratolytically active substances, etc.

Corresponding requirements apply mutatis mutandis to the formulation of medicinal preparations.

Medicinal topical compositions for the purposes of the present invention generally contain one or more medicaments in an effective concentration. For the sake of simplicity, for a clear distinction between cosmetic and medicinal application and corresponding products, reference is made to the legal provisions of the Federal Republic of Germany (e.g., Cosmetics Directive, Food and Drugs Act).

In this connection it is likewise advantageous to add the active substance used according to the invention as an additive to preparations that already contain other active substances for other purposes.

Accordingly, for the purposes of the present invention, cosmetic or topical dermatological compositions can be used, depending on their structure, e.g., as skin protection cream, cleansing milk, sunscreen lotion, nourishing cream, day or night cream, etc. It is optionally possible and advantageous to use the compositions according to the invention as a basis for pharmaceutical formulations.

However, for the purposes of the present invention it is also advantageous to produce cosmetic and dermatological preparations the main purpose of which is not to protect against sunlight, but which nevertheless have a content of UV protective substances. Thus, for example, UVA and UVB filter substances are usually incorporated into day creams.

Preparations according to the invention can advantageously contain substances that absorb UV radiation in the UVB range, the total amount of the filter substances being, e.g., 0.1% by weight to 30% by weight, preferably 0.5 to 10% by weight, in particular 1 to 6% by weight, based on the total weight of the preparations.

The UVB filters can be oil-soluble or water-soluble. Examples of oil-soluble substances are:
3-benzylidenecamphor und derivatives thereof, e.g., 3-(4-methylbenzylidene)-camphor,
4-aminobenzoic acid derivatives, preferably 2-ethylhexyl 4-(dimethylamino)-benzoate, amyl 4-(dimethylamino)benzoate;
esters of cinnamic acid, preferably 2-ethylhexyl 4-methoxycinnamate, isopentyl 4-methoxycinnamate;
esters of salicylic acid, preferably 2-ethylhexyl salicylate, 4-isopropylbenzyl salicylate, homomenthyl salicylate;
derivatives of benzophenone, preferably 2-hydroxy-4-methoxybenzophenone, 2-hydroxy-4-methoxy-4′-methylbenzophenone, 2,2′-dihydroxy-4-methoxybenzo-phenone;
esters of benzalmalonic acid, preferably di(2-ethylhexyl) 4-methoxybenzal-malonate;
2,4,6-trianilino-(p-carbo-2′-ethyl-1′-hexyloxy)-1,3,5-triazine.

Advantageous water-soluble substances are:
2-phenylbenzimdazole-5-sulfonic acid and salts thereof, e.g., sodium, potassium or triethanolammonium salts,
sulfonic acid derivatives of benzophenones, preferably 2-hydroxy-4-methoxybenzophenone-5-sulfonic acid and the salts thereof;
sulfonic acid derivatives of 3-benzylidene camphor, such as, e.g., 4-(2-oxo-3-bornylidenemethyl)benzene sulfonic acid, 2-methyl-5-(2-oxo-3-bornylidenemethyl)sulfonic acid and the salts thereof.

Of course, the list of the referenced UVB filters that can be used according to the invention is not intended to be limiting.

The subject of the invention is also the combination of a UVA filter according to the invention with a UVB filter or a cosmetic or dermatological preparation according to the invention which also contains a UVB filter.

It can also be advantageous to use UVA filters that are customarily present in cosmetic and/or dermatological preparations in preparations according to the invention. Such filter substances are preferably derivatives of dibenzoylmethane, in particular 1-(4′-tert-butylphenyl)-3-(4′-methoxyphenyl)-propane-1,3-dione and 1-phenyl-3-(4′-isopropyl-phenyl)-propane-1,3-dione. Preparations that contain these combinations are also the subject of the invention. The same amounts of UVA filter substances can be used as have been given for UVB filter substances.

Cosmetic and/or dermatological preparations for the purposes of the present invention can also contain inorganic pigments that are customarily used in cosmetics for protecting the skin against UV rays. These are oxides of titanium, zinc, iron, zirconium, silicon, manganese, aluminum, cerium and mixtures thereof, and modifications in which the oxides are the active agents. Particular preference is given to pigments based on titanium dioxide. The amounts given for the above combinations can be used.

The cosmetic and dermatological preparations according to the invention can contain cosmetic active ingredients, auxiliaries and/or additives as are customarily used in such preparations, e.g., antioxidants, preservatives, bactericides, perfumes, antifoaming substances, dyes, pigments that have a coloring effect, thickeners, surfactants, emulsifiers, emollients, moisturizers and/or humectants, fats, oils, waxes or other customary constituents of a cosmetic or dermatological formulation, such as alcohols, polyols, polymers, foam stabilizers, electrolytes, organic solvents or silicone derivatives.

If the cosmetic or dermatological preparation for the purposes of the present invention is a solution or an emulsion or a dispersion, solvents that can be used are:
water or aqueous solutions
oils, such as triglycerides of capric or caprylic acid, but preferably castor oil;
fats, waxes and other natural and synthetic fatty substances, preferably esters of fatty acids with alcohols of low carbon number, e.g., with isopropanol, propylene glycol or glycerin, or esters of fatty alcohols with alkanoic acids of low carbon number or with fatty acids;
alcohols, diols or polyols of low carbon number, and ethers thereof, preferably ethanol, isopropanol, propylene glycol, glycerin, ethylene glycol, ethylene glycol monoethyl or monobutyl ether, propylene glycol monomethyl, monoethyl or monobutyl ether, diethylene glycol monomethyl or monoethyl ether and analogous products.

In particular, mixtures of the above-mentioned solvents are used. In the case of alcoholic solvents, water can be a further constituent.

The oil phase of the emulsions, oleogels or hydrodispersions or lipodispersions for the purposes of the present invention is advantageously chosen from the group of esters of saturated and/or unsaturated, branched and/or unbranched alkanecarboxylic acids having a chain length of 3 to 30 carbon atoms and saturated and/or unsaturated, branched and/or unbranched alcohols having a chain length of 3 to 30 carbon atoms, from the group of esters of aromatic carboxylic acids and saturated and/or unsaturated, branched and/or unbranched alcohols having a chain length of 3 to 30 carbon atoms. Such ester oils can then advantageously be chosen from the group of isopropyl myristate, isopropyl palmitate, isopropyl stearate, isopropyl oleate, n-butyl stearate, n-hexyl laurate, n-decyl oleate, isooctyl stearate, isononyl stearate, isononyl isononanoate, 2-ethylhexyl palmitate, 2-ethylhexyl laurate, 2-hexyldecyl stearate, 2-octyldodecyl palmitate, oleyl oleate, oleyl erucate, erucyl oleate, erucyl erucate, and synthetic, semi-synthetic and natural mixtures of such esters, e.g., jojoba oil.

Furthermore, the oil phase can be advantageously chosen from the group of branched and unbranched hydrocarbons and hydrocarbon waxes, silicone oils, dialkyl ethers, the group of saturated or unsaturated, branched or unbranched alcohols, and fatty acid triglycerides, namely the triglycerol esters of saturated and/or unsaturated, branched and/or unbranched alkanecarboxylic acids having a chain length of 8 to 24, in particular 12-18 carbon atoms. The fatty acid triglycerides can, for example, be advantageously chosen from the group of synthetic, semi-synthetic and natural oils, e.g., olive oil, sunflower oil, soya oil, peanut oil, rapeseed oil, almond oil, palm oil, coconut oil, palm kernel oil and the like.

Any blends of such oil and wax components can also be used advantageously for the purposes of the present invention. In some cases it can also be advantageous to use waxes, e.g., cetyl palmitate, as the sole lipid component of the oil phase.

The oil phase is advantageously chosen from the group of 2-ethylhexyl isostearate, octyldodecanol, isotridecyl isononanoate, isoeicosane, 2-ethylhexyl cocoate, C₁₂₋₁₅-alkyl benzoate, caprylic/capric acid triglyceride, dicaprylyl ether.

Particularly advantageous are mixtures of C₁₂₋₁₅-alkyl benzoate and 2-ethylhexyl isostearate, mixtures of C₁₂₋₁₅-alkyl benzoate and isotridecyl isononanoate and mixtures of C₁₂₋₁5-alkyl benzoate, 2-ethylhexyl isostearate and isotridecyl isononanoate.

Of the hydrocarbons, paraffin oil, squalane and squalene may be used advantageously for the purposes of the present invention.

Advantageously, the oil phase can also have a content of cyclic or linear silicone oils or it can entirely consist of such oils, although it is preferred to use an additional content of other oil phase components apart from the silicone oil or the silicone oils.

Cyclomethicone (octamethyl cyclotetrasiloxane) is advantageously used as the silicone oil to be used according to the invention. However, other silicone oils can also be used advantageously for the purposes of the present invention, e.g., hexamethylcyclotrisiloxane, polydimethylsiloxane, poly(methylphenylsiloxane).

Mixtures of cyclomethicone and isotridecyl isononanoate, of cyclomethicone and 2-ethylhexyl isostearate are also particularly advantageous.

The aqueous phase of the preparations according to the invention optionally advantageously contains:
alcohols, diols or polyols of low C number, and ethers thereof, preferably ethanol, isopropanol, propylene glycol, glycerol, ethylene glycol, ethylene glycol monoethyl or monobutyl ether, propylene glycol monomethyl, monoethyl or monobutyl ether, diethylene glycol monomethyl or monoethyl ether and analogous products, and alcohols of low C number, e.g., ethanol, isopropanol, 1,2-propanediol, glycerin and in particular one or more thickeners that can advantageously be chosen from the group of silicon dioxide, aluminum silicates, polysaccharides or derivatives thereof, e.g., hyaluronic acid, xanthan gum, hydroxypropylmethylcellulose, particularly advantageously from the group of polyacrylates, preferably a polyacrylate from the group of so-called carbopols, e.g., carbopol grades 980, 981, 1382, 2984, 5984, in each case singly or in combination.

Customary bases that are suitable for use as cosmetic sticks for the purposes of the present invention are liquid oils (e.g., paraffin oils, castor oil, isopropyl myristate), semisolid constituents (e.g., Vaseline, lanolin), solid constituents (e.g., beeswax, ceresine and microcrystalline waxes or ozokerite) and high-melting waxes (e.g., carnauba wax and candelilla wax)

Suitable propellants for cosmetic and/or dermatological O/W emulsions that can be sprayed from aerosol containers for the purposes of the present invention are the customary known, readily volatile, liquefied propellants, e.g., hydrocarbons (propane, butane, isobutane), which can be used singly or in a mixture with one another. Compressed air may also be used advantageously.

Of course, one of skill in the art is aware that there are propellant gases which are nontoxic per se and would in principle be suitable for realizing the present invention in the form of aerosol preparations, but which nevertheless should be avoided because of their unacceptable impact on the environment or other accompanying circumstances, in particular fluorocarbons and chlorofluorocarbons (CFCs).

For the purposes of the present invention, cosmetic preparations can also be present in the form of gels which, in addition to an effective content of the active substance according to the invention and solvents customarily used therefor, preferably water, also contain organic thickeners, e.g., gum arabic, xanthan gum, sodium alginate, cellulose derivatives, preferably methylcellulose, hydroxymethylcellulose, hydroxyethylcellulose, hydroxypropylcellulose, hydroxypropylmethylcellulose, or inorganic thickeners, e.g., aluminum silicates, such as, e.g., bentonites, or a mixture of polyethylene glycol and polyethylene glycol stearate or distearate. The thickener is present in the gel, e.g., in an amount between 0.1 and 30% by weight, preferably between 0.5 and 15% by weight.

O/W Emulsions

| | Examples | | | | | | | | | |
|---|---|---|---|---|---|---|---|---|---|---|
| | 1 | 2 | 3 | 4 | 5 | 6 | 7 | 8 | 9 | 10 |
| Glycerin monostearate SE | 0.50 | 1.00 | 3.00 | | | 2.50 | | | | |
| Glyceryl stearate citrate | 2.00 | | | 1.00 | 2.00 | | 2.50 | | 3.00 | |
| Stearic acid | | 3.00 | 0.75 | 2.00 | | | | | | |
| Sorbitan stearate | | | | | | | | | 1.00 | |
| Polyglyceryl methylglucose | | | | | | | | 3.00 | | |
| distearate | | | | | | | | | | |
| Ceylsttearyl alcohol, PEG-40 | | | | | | | | | | 4.00 |
| castor oil, sodium cetylstearyl | | | | | | | | | | |
| sulfate | | | | | | | | | | |
| PEG-40 stearate | 0.50 | | | | | 1.10 | | | | |
| PEG-100 stearate | | | 1.50 | | | | | | | |
| Laurylmethicone copolyol | | | | 0.75 | | 0.50 | | | | |
| Cetyl phosphate | | | 0.75 | | 1.00 | | | | | |
| Stearyl alcohol | | | 3.00 | | | 4.00 | 0.50 | 1.00 | | 2.00 |
| Cetyl alcohol | 2.50 | 1.00 | | | 0.50 | | 2.00 | 1.00 | 2.00 | |
| Bis-ethylhexyloxyphenol | | | | | | | | | | 1.5 |
| methoxyphenyl triazine | | | | | | | | | | |
| Diethylaminohydroxybenzoyl- | | | 2.0 | | | | | | | |
| hexylbenzoate | | | | | | | | | | |
| Titanium dioxide T 805 | | 2.0 | | | | | | | | |
| C12-15 alkyl benzoate | | 2.50 | | | | 7.00 | 5.00 | 3.00 | 6.00 | |
| Dicaprylyl ether | | | 3.50 | | 2.00 | | | 3.00 | 2.00 | |
| Butylene glycol dicaprylate/dicaprate | 5.00 | | | 5.00 | 3.00 | | | | 1.00 | 3.00 |
| Cetylstearyl isononanoate | | 4.00 | | | | 2.00 | 2.00 | 2.00 | | |
| Dimethicone | | 0.50 | 1.00 | | 2.00 | | | 2.00 | | 2.00 |
| Cyclomethicone | 2.00 | | | 4.50 | | | 0.50 | | 3.00 | 3.00 |
| Dimethicone/vinyl | | 4.00 | | | | | 0.50 | | | |
| dimethicone crosspolymer | | | | | | | | | | |
| PVP hexadecene copolymer | 0.50 | | | 0.50 | 1.00 | | 1.00 | | | |
| Glycerin | 3.00 | 7.50 | | 7.50 | 5.00 | | 2.50 | 10.0 | 12.00 | 8.00 |
| Xanthan gum | 0.15 | | 0.05 | | | | 0.30 | | | |
| Sodium carbomer | | | | | | | | 0.20 | 0.30 | 0.2 |
| Glycerin | | 5.00 | | | | 7.00 | | | | |
| Vitamin E acetate | 0.50 | | 0.25 | 0.50 | 0.75 | | 1.00 | | 0.50 | 0.50 |
| Ubiquinones | 0.25 | 0.10 | 0.05 | 0.20 | 0.003 | 0.10 | 0.04 | 0.10 | 0.15 | 0.08 |
| Glycine soya | 0.05 | 0.30 | 1.50 | 1.00 | 1.00 | 2.00 | 1.8 | 1.00 | 3.00 | 0.9 |
| Creatine | 0.10 | | | 1.00 | | | 0.50 | | 0.30 | |
| 1-methylhydantoin-2-imide | 0.01 | | | 0.40 | | | 0.20 | | 0.10 | |
| DMDM hydantoin | | 0.60 | | 0.20 | | | | | 0.20 | |
| Methylparaben | 0.15 | | 0.25 | | 0.50 | 0.28 | | 0.20 | | 0.30 |
| Propylparaben | | | | | | 0.14 | | | | |
| Phenoxyethanol | 1.00 | 0.40 | | 0.40 | 0.50 | 0.50 | 0.60 | 0.50 | | 0.40 |
| EDTA | | 0.20 | 0.35 | 0.50 | 0.02 | | 0.03 | | | |
| Ethanol | | 2.00 | 1.50 | | 3.00 | 5.00 | 1.00 | | | |
| Perfume | 0.20 | 0.20 | | | | 0.30 | 0.40 | | 0.25 | 0.30 |
| Water | ad 100 | ad 100 | ad 100 | ad 100 | ad 100 | ad 100 | ad 100 | ad 100 | ad 100 | ad 100 |

Glycine soya = Isoflavone 150 from Lucas Meyer

| | Foam-like O/W emulsions: | | | |
|---|---|---|---|---|
| | Example 11 | | Example 12 | |
| | % by | % by | % by | % by |
| | weight | vol. | weight | vol. |
| Stearic acid | 5.00 | | 1.00 | |
| Cetyl alcohol | 5.50 | | | |
| Cetyl stearyl | | | 2.00 | |
| alcohol | | | | |
| PEG-40 stearate | 8.50 | | | |
| PEG-20 stearate | | | 1.00 | |
| Caprylic acid/capric | 4.00 | | 2.00 | |
| acid triglyceride | | | | |
| C12-15 alkyl | 10.00 | | 15.50 | |
| benzoate | | | | |
| Cyclomethicone | 4.00 | | | |
| Dimethicone | | | 0.50 | |
| Octyl isostearate | | | 5.00 | |
| Myristyl myristate | | | 2.00 | |
| Ceresine | 1.50 | | | |
| Glycerin | | | 3.00 | |
| Phenylbenzimidazole | | | 1.0 | |
| sulfonic acid | | | | |
| Butylmethoxydibenzoyl- | | | 2.0 | |
| methane | | | | |
| Ethylhexyltriazone | 1.5 | | | |
| BHT | | | 0.02 | |
| Na₂H₂EDTA | 0.50 | | 0.10 | |
| Ubiquinone | 1.00 | | 0.20 | |
| Glycine soya | 0.10 | | 0.80 | |
| Creatine | | | 0.80 | |
| 1-methylhydantoine- | | | 0.30 | |
| 2-imide | | | | |
| Perfume, preservatives | q.s. | | q.s. | |
| Dyes, etc. | q.s. | | q.s. | |
| Potassium hydroxide | q.s. | | q.s. | |
| Water | ad 100.00 | | ad 100.00 | |
| | pH value to | | pH value to | |
| | 6.5-7.5 | | 5.0-6.0 | |
| Emulsion 1 | | 70 | | |
| Emulsion 2 | | | | 35 |
| Gas (nitrogen) | | 30 | | |
| Gas (helium) | | | | 65 |

Combination of the fatty/light-protective filter phase heated to 78° C. with the aqueous/light-protective filter phase heated to 75° C. Homogenization by means of a toothed-rim dispersing machine (rotor-stator principle) at 65° C. Stirring for 45 min. in Becomix with gassing with helium at 1 bar and cooling to 30° C. Addition of the additives at 30° C. (perfume). Homogenization by means of a toothed-rim dispersing machine (rotor-stator principle) at 23° C.

1-7. (canceled)

8. A method of at least one of tightening and firming skin, wherein the method comprises applying to at least a part of the skin a cosmetic or dermatological O/W emulsion which comprises one or more bioquinones and one or more isoflavonoids.

9. The method of claim 8, wherein the one or more bioquinones comprise coenzyme Q10.

10. The method of claim 8, wherein the one or more isoflavonoids comprise genistein.

11. The method of claim 9, wherein the one or more isoflavonoids comprise genistein.

12. The method of claim 8, wherein the one or more isoflavonoids are present as a part of a soy extract.

13. The method of claim 12, wherein the soy extract comprises from 5% to 20% by weight of saponins, based on a total weight of the soy extract.

14. The method of claim 13, wherein the soy extract comprises from 10% to 18% by weight of saponins.

15. The method of claim 14, wherein the soy extract comprises genistein.

16. The method of claim 8, wherein the one or more bioquinones are present in an amount of from 0.000001% to 5% by weight, based on a total weight of the O/W emulsion.

17. The method of claim 8, wherein the one or more isoflavonoids are present in an amount of from 0.001% to 10% by weight, based on a total weight of the O/W emulsion.

18. The method of claim 8, wherein the one or more isoflavonoids are present in an amount of from 0.01% to 1% by weight, based on a total weight of the O/W emulsion.

19. The method of claim 8, wherein a weight ratio of the one or more isoflavonoids and the one or more bioquinones is from 2:1 to 1:2.

20. The method of claim 8, wherein the O/W emulsion further comprises saponins in an amount of from 0.001% to 2% by weight, based on a total weight of the O/W emulsion.

21. The method of claim 20, wherein the saponins are present in an amount of from 0.02% to 0.04% by weight.

22. A method for at least one of the treatment and prophylaxis of cellulite, wherein the method comprises applying to at least a part of the skin a cosmetic or dermatological O/W emulsion which comprises one or more bioquinones and one or more isoflavonoids.

23. The method of claim 22, wherein the one or more bioquinones comprise coenzyme Q10.

24. The method of claim 22, wherein the one or more isoflavonoids comprise genistein.

25. The method of claim 23, wherein the one or more isoflavonoids comprise genistein.

26. The method of claim 22, wherein the one or more isoflavonoids are present as a part of a soy extract.

27. The method of claim 26, wherein the soy extract comprises from 5% to 20% by weight of saponins, based on a total weight of the soy extract.

28. The method of claim 27, wherein the soy extract comprises from 10% to 18% by weight of saponins.

29. The method of claim 28, wherein the soy extract comprises genistein.

30. The method of claim 22, wherein the one or more bioquinones are present in an amount of from 0.000001% to 5% by weight, based on a total weight of the O/W emulsion.

31. The method of claim 22, wherein the one or more isoflavonoids are present in an amount of from 0.001% to 10% by weight, based on a total weight of the O/W emulsion.

32. The method of claim 22, wherein the one or more isoflavonoids are present in an amount of from 0.01% to 1% by weight, based on a total weight of the O/W emulsion.

33. The method of claim 22, wherein a weight ratio of the one or more isoflavonoids and the one or more bioquinones is from 2:1 to 1:2.

34. The method of claim 22, wherein the O/W emulsion further comprises saponins in an amount of from 0.001% to 2% by weight, based on a total weight of the O/W emulsion.

35. The method of claim 34, wherein the saponins are present in an amount of from 0.02% to 0.04% by weight.

36. A dermatological or cosmetic O/W emulsion, wherein the emulsion comprises one or more bioquinones and one or more isoflavonoids.

37. The emulsion of claim 36, wherein the one or more bioquinones comprise at least coenzyme Q10 and the one or more isoflavonoids comprise at least genistein.

38. The emulsion of claim 36, wherein the one or more bioquinones are present in an amount from 0.000001% to 5% by weight and the one or more isoflavonoids are present in an amount of from 0.01% to 1% by weight, based on a total weight of the emulsion.

39. The emulsion of claim 38, wherein a weight ratio of the one or more isoflavonoids and the one or more bioquinones is from 10:1 to 1:50.

40. The emulsion of claim 36, wherein the emulsion further comprises saponins in an amount of from 0.001% to 2% by weight, based on a total weight of the emulsion.

41. The emulsion of claim 38, wherein the emulsion further comprises saponins in an amount of from 0.02% to 0.04% by weight, based on a total weight of the emulsion.

42. The emulsion of claim 36, wherein the one or more isoflavonoids are present as a part of a soy extract.

43. The emulsion of claim 42, wherein the soy extract comprises from 5% to 20% by weight of saponins, based on a total weight of the soy extract.

44. The emulsion of claim 43, wherein the soy extract comprises from 10% to 18% by weight of saponins.

45. The emulsion of claim 43, wherein the soy extract comprises genistein.

46. The emulsion of claim 37, wherein the one or more bioquinones are present in an amount of from 0.000001% to 5% by weight and the one or more isoflavonoids are present in an amount of from 0.001% to 10% by weight, based on a total weight of the emulsion, and wherein the emulsion further comprises saponins in an amount of from 0.001% to 2% by weight, based on a total weight of the emulsion.

47. The emulsion of claim 46, wherein a weight ratio of the one or more isoflavonoids and the one or more bioquinones is from 2:1 to 1:2.

48. The emulsion of claim 46, wherein the one or more isoflavonoids are present as a part of a soy extract.

49. The emulsion of claim 46, wherein the one or more isoflavonoids are present in an amount of from 0.01% to 1% by weight, based on a total weight of the emulsion, and wherein the emulsion further comprises saponins in an amount of from 0.02% to 0.04% by weight, based on a total weight of the emulsion.
